(19) United States
(12) Patent Application Publication
(10) Pub. No.: US 2023/0087344 A1
(43) Pub. Date: Mar. 23, 2023

(54) Mattress With X-Ray Sleeve And Method Of Use Thereof

(71) Applicant: LINET SPOL. S R.O., Slany (CZ)

(72) Inventor: Martin Toms, Hampshire (GB)

(73) Assignee: LINET SPOL. S R.O., Slany (CZ)

(21) Appl. No.: 17/479,178

(22) Filed: Sep. 20, 2021

(51) Int. Cl. A47C 21/02; A47C 31/10; A61B 6/04; A61B 6/00

(52) U.S. Cl. CPC A47C 21/022; A47C 31/105; A61B 6/0407; A61B 6/4423; A61B 6/50

(57) ABSTRACT

An X-ray sleeve is configured to be releasably attached a mattress cover. The sleeve is configured to receive an X-ray cassette, protected from foreign substances, such as against contaminants, such as bodily fluids, and can be removeable for sterilization and sanitation.

### BACKGROUND OF THE INVENTION

This invention relates in general to beds and more particularly to mattresses for medical or hospital beds. Most particularly, the invention relates to a mattress cover with an inner sleeve for receiving an X-ray cassette and method of use thereof.

The chest radiography or X-ray for patients confined to beds is a critical tool for monitoring, diagnosing and treating conditions of patients in an intensive care unit (ICU) who may be difficult to position or unable to cooperate. The chest X-ray can reveal conditions that may not be otherwise clinically detected. The chest X-ray may be used to produce images of the heart, lungs, airways, blood vessels and bones of the chest and spine, to detect, diagnose and monitor cardiopulmonary conditions and complications, such as shortness of breath, persistent cough, chest pain, or conditions related to injury, and treatment for conditions, such as pneumonia, emphysema and cancer. To accommodate chest X-rays, the back rest portion of bed decks, as well as the mattress and mattress cover, are often formed of a material that does not interfere with electromagnetic waves of the X-ray equipment. However, beds must also take into consideration cleanliness, as well as sterilization and sanitation.

### SUMMARY OF THE INVENTION

This invention relates to an X-ray sleeve that is configured to be releasably attached a mattress cover, wherein the sleeve is configured to receive an X-ray cassette, protected from foreign substances, such as against contaminants, such as bodily fluids, and can be removeable for sterilization and sanitation.

The mattress cover comprises a top cover comprising an upper panel defining an upper surface for supporting a patient. The upper panel may comprise opposingly disposed, laterally extending edges and opposingly disposed, longitudinally extending edges, which may, at least, in part, cooperatively define a peripheral edge (i.e., an edge about the periphery of the upper panel). A peripheral panel may depend downwardly in relation to the peripheral edge of the upper panel. The peripheral panel may comprise, at least, in part, opposingly disposed, laterally extending panel portions and opposingly disposed, longitudinally extending panel portions. An inner panel may depend downwardly in relation to the upper panel and interiorly or inwardly of the peripheral panel. At least a portion of the inner panel may define a longitudinally extending slit, passage or opening supported in relation to at least a portion of at least one of the longitudinally extending edges of the upper panel, preferably at a head end of the mattress cover. A longitudinally extending closure (i.e., an outer closure) may be operable to a closed position to close the opening and an open position to open the opening. A longitudinally extending closure portion may be provided within the opening. The longitudinally extending closure portion may be comprised of at least an upper inner first longitudinally extending closure portion. The sleeve may be configured to be extendable laterally beneath the upper panel. The sleeve may be dimensioned and configured to receive an X-ray cassette. The sleeve may comprise a longitudinally extending closure portion that mates with the longitudinally extending closure portion within the opening to attach a longitudinally extending lateral portion of the sleeve to the top cover within the opening. The longitudinally extending closure portion of the sleeve may include an upper second longitudinally extending closure portion that mates with the upper inner first longitudinally extending closure portion to attach an upper longitudinally extending lateral portion of the sleeve to the top cover. The outer closure when in the closed position protects the sleeve from foreign substances, such as against contaminants, such as bodily fluids, and permits access to the X-ray sleeve for permitting insertion of the X-ray cassette therein, and the closure portion of the sleeve permits the sleeve to be attached and removed for sterilization and sanitation.

Various advantages of this invention will become apparent to those skilled in the art from the following detailed description of the preferred embodiment, when read in light of the accompanying drawings.

### BRIEF DESCRIPTION OF THE DRAWINGS

Various features and attendant advantages of the present invention will become more fully appreciated as the same becomes better understood when considered in conjunction with the accompanying drawings, in which like reference characters designate the same or similar parts throughout the several views, and wherein:

FIG. 1 is an exploded perspective view of an exemplary X-ray sleeve supported in relation to an exemplary mattress cover,

FIG. 2 is an enlarged side elevational view of a portion of an inner panel of the cover with an outer closure to provide access to an outer closure in an open position to provide access to a slit, passage or opening, and more particularly, an X-ray sleeve within the opening for insertion and retrieval of an X-ray cassette into and from the X-ray sleeve, respectively;

FIG. 3 is an enlarged side elevational view of the portion of the inner panel shown in FIG. 2 further showing closure portions of the sleeve that are operable to a closed position to prevent the sleeve from being removed for the inner panel of the cover;

FIG. 4 is an enlarged side elevational view of the portion of the inner panel shown in FIG. 3 further showing closure portions of the sleeve that are operable to an opened position to permit the sleeve to be removed for the inner panel of the cover for sterilization and sanitation purposes;

FIGS. 5A-5C are diagrammatic representations of methods for using an X-ray cassette.

The drawings are diagrammatic representations rather than literal depictions of their content, do not purport to show all structure or every component that would be present in a real-world model, and are not necessarily drawn to scale.

### DETAILED DESCRIPTION OF THE PREFERRED EMBODIMENT

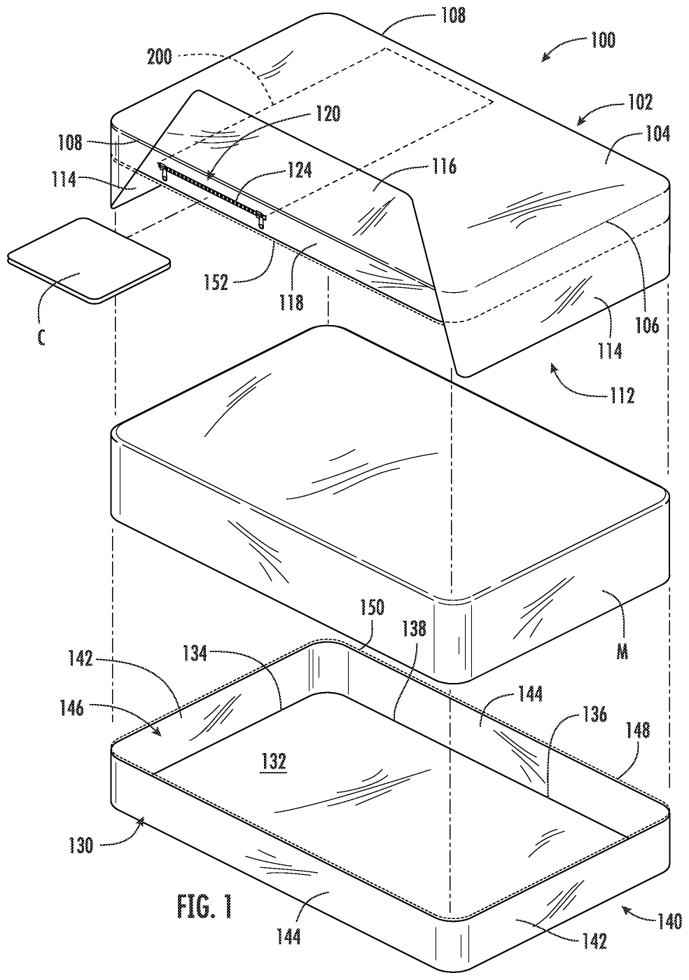

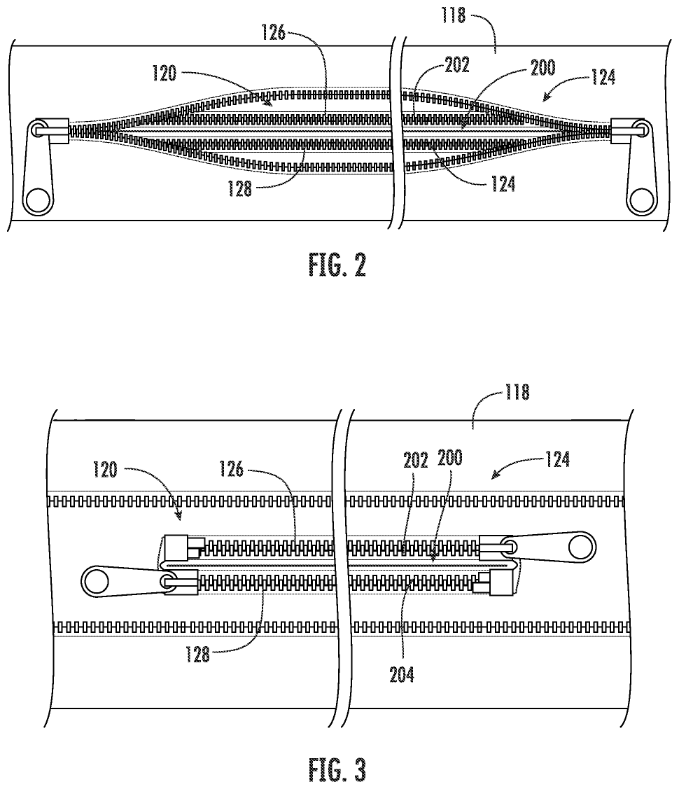

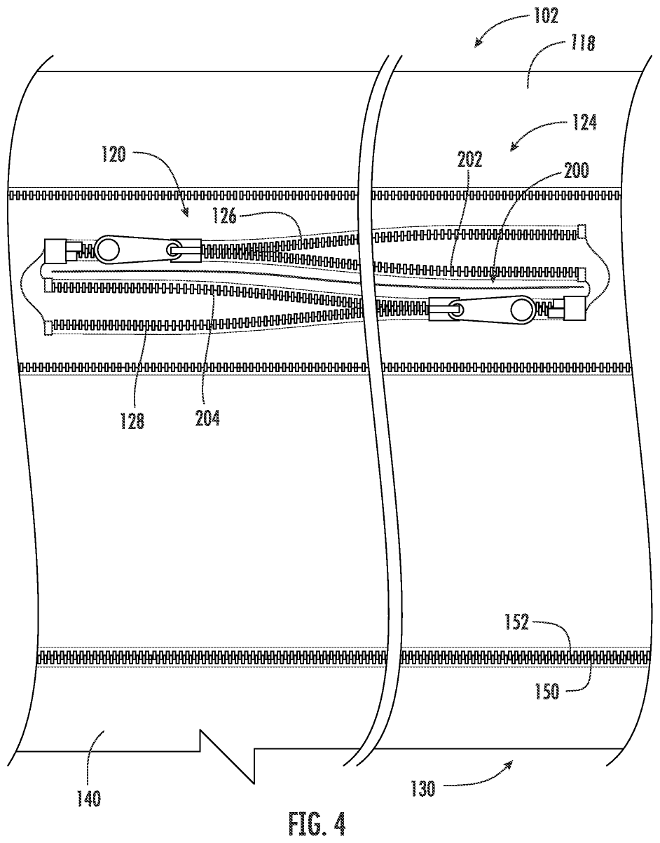

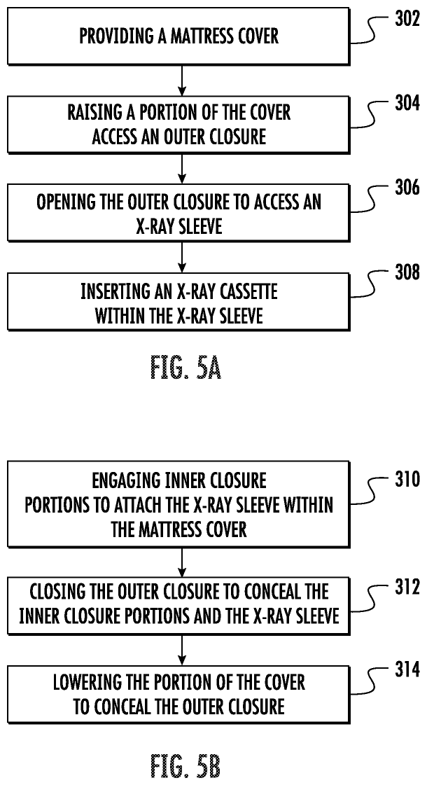

Referring now to the drawings, there is illustrated in FIGS. 1-4 a mattress cover 100 and an X-ray sleeve 200 that is configured to be releasably attached to the mattress cover 100 for receiving an X-ray cassette C, to protect the X-ray cassette C from bodily fluids, and which can be removeable for sterilization and sanitation thereof. The mattress cover 100 and sleeve 200 may be formed from any suitable fabric. The fabric is preferably water repellent, highly durable and mildew resistant. The fabric may be a polyester fabric, which may be made waterproof by a vinyl material, such as a poly-coating (e.g., a polyurethane (PU) coating), for example, on one or more surfaces and/or layers thereof. The X-ray sleeve 200 may be sized or dimensioned to receive cassettes of any suitable size. The cassette may be a conventional film cassette, as used in a conventional radiography process, or a Phosphor Imaging Plate, as used in computed radiography.

The mattress cover 100 may be comprised of a top cover 102, which may be comprised of an upper panel 104 defining an upper surface for supporting a patient. The upper panel 104 may comprise opposingly disposed, laterally extending edges 106 and opposingly disposed, longitudinally extending edges 108, at least, in part, cooperatively defining a peripheral edge 110. A peripheral panel 112 may depend downwardly in relation to the peripheral edge 110 of the upper panel 104. The peripheral panel 112 may comprise at least, in part, opposingly disposed, laterally extending panel portions 114 and opposingly disposed, longitudinally extending panel portions 116. An inner panel 118 may depend downwardly in relation to the upper panel 104 and inwardly of the peripheral panel 112. At least a portion of the inner panel 118 may define a longitudinally extending slit, passage or opening 120, generally at reference numeral 120, supported in relation to at least a portion of at least one of the longitudinally extending edges 108 of the upper panel 104 and a lower peripheral edge 122. An opening 120 may be supported in relation to at least a portion of at least one of the longitudinally extending edges 108 of the upper panel 104 and a lower peripheral edge 122. The peripheral panel 112 may function may as a skirt for concealing the inner panel 118, as well as the opening 120 to protect the opening 120 from contaminants. The peripheral panel 112 and the inner panel 118 may be supported in relation to the upper panel 104, about the peripheral edge 110 thereof, in any suitable manner, such as but stitching and/or welding techniques. The peripheral panel 112 may be folded over itself to form a double layer skirt with a PU coating on an outer surface of each layer.

A longitudinally extending outer closure 124 may be operable to a closed position to close the opening 120 and an open position to open the opening 120. An inner longitudinally extending closure portion may be provided within the opening. In the illustrated embodiment, at least an upper inner first longitudinally extending closure portion 126 is provided within the opening 120. The X-ray sleeve 200 may be extendable laterally beneath the upper panel 104. The X-ray sleeve 200 is dimensioned and configured to receive the X-ray cassette C to support the X-ray cassette C beneath the upper panel 104 of the top cover 102, such as between the upper panel 104 and a mattress M there below. The X-ray sleeve 200 may comprise an upper second longitudinally extending closure portion 202 that mates with the upper inner first longitudinally extending closure portion 126 to releasably attach an upper longitudinally extending lateral portion 204 of the sleeve 200 to the inner panel 118 of the top cover 102 within the opening 120. The outer closure 124, when in the closed position, protects the X-ray sleeve 200 from foreign substances, such as against contaminants, such as bodily fluids, and permits access to the X-ray sleeve 200 for permitting insertion of the X-ray cassette C therein, and the closure portion 202 of the X-ray sleeve 200 mates with the closure portion 126 to permit the X-ray sleeve 200 to be removed for sterilization and sanitation.

The top cover 100 may further comprise a lower inner first longitudinally extending closure portion 128 within the opening 120 and juxtaposed the upper inner first longitudinally extending closure portion 126, and the X-ray sleeve 200 may further comprise a lower second longitudinally extending closure portion 204 that mates with the lower inner first longitudinally extending closure portion 128 to releasably attach a lower longitudinally extending lateral portion 208 of the X-ray sleeve 200 to the inner panel 118 of the top cover 100 within the opening 120.

At least a portion of the peripheral panel 112 is preferably pliable so that at least a portion of the peripheral panel 112 may be lifted or raised so that at least a portion of the peripheral panel 112 is above or otherwise out of the way of the opening 102 to expose the opening 120 otherwise concealed by the peripheral panel 112. The opening 120 is preferably concealed by the peripheral panel 112 when the peripheral panel 112 drapes downwardly from the upper panel 104. In this manner, the peripheral panel 112 functions as a skirt.

The longitudinally extending outer closure 124 may be in the form of a zipper, and may be in the form of a two-way zipper, and each one of the longitudinally extending closure portions 126, 128 and 202, 204 are preferably zipper portions, which cooperatively form zippers.

It should be appreciated that the zippers may include tape, which may be attached, such as be stitching, to edges of the inner panel 118 about the opening 120 therein and to edges of the sleeve 200. Edges of the tape may support elements or teeth, which cooperate with a slider or head to engage the teeth to close the zippers and disengage the teeth to open the zippers. Stops may be provided at each end of the outer closure 124 to limit travel of the slider. The zipper portions forming the inner closure portions 202, 204 supported in relation to edges of the sleeve 200 may have a box at one end of the tape and a stop at an opposing end of the tape to limit travel of the slider. The zipper portions forming the inner closure portions 126, 128 supported in relation to edges of the inner panel 118 about the opening 120 may have a pin at one end of the tape that is engageable with the slider and the box for engagement of the teeth of the zipper portions and a stop at an opposing end of the tape to limit travel of the slider.

It should be appreciated that the closures and closure portions may be some other suitable portions, such as mating hook and loop type fasteners or fastener portions, buttons, and snap fasteners.

The mattress cover 100 may further be comprised of a bottom cover 130, which may be comprised of a lower panel 132 that may comprise opposingly disposed, laterally extending edges 134 and opposingly disposed, longitudinally extending edges 136 that, at least, in part, may cooperatively define a peripheral edge 138. A peripheral panel 140 may extend upwardly in relation to the peripheral edge 138 of the lower panel 132. The peripheral panel 140 of the bottom cover 130 may comprise, at least, in part, opposingly disposed, laterally extending panel portions 142 and opposingly disposed, longitudinally extending panel portions 144. The lower panel 132 and the bottom cover peripheral panel 140 may cooperatively define a partial lower enclosure, generally viewed at 146, for a mattress M. At least one bottom cover closure portion 148 may be supported in relation to the bottom cover peripheral panel 140, preferably in relation to an upper peripheral edge 150 of the bottom cover peripheral panel 140. The top cover 102 may further comprise at least one top cover closure portion 150, which may be supported in relation to the top cover peripheral panel 112, preferably in relation to the lower peripheral edge 122 of the inner panel 118. The top cover closure portion 150 may mate with the bottom cover closure portion 148 to attach the top cover 102 to the bottom cover 130 to cover or enclose the mattress M therein. It should also be appreciated that the peripheral panel 112 of the top cover 102 may function to form a skirt to cover the peripheral panel 140 of the bottom cover 130, while at the same time covering the inner panel 118 and the opening 120.

The closure portions 148, 150 may cooperatively defined a zipper, which may be comprised to tape, which may be attached, such as be stitching, to edges of the upper peripheral edge 150 of the bottom cover peripheral panel 140 and the lower peripheral edge 122 of the inner panel 118. Edges of the tape may support elements or teeth, which cooperate with a slider or head to engage the teeth to close the zipper and disengage the teeth to open the zipper. One of the zipper portions 148, 150 forming may have a pin at one end of the tape that is engageable with the slider and the box for engagement of the teeth of the zipper portions and a stop at an opposing end of the tape to limit travel of the slider. This arrangement permit the closure portions 148, 150 to separate, which permit the top cover 102 to be separated from the bottom cover 130.

It should be appreciated that the closure portions 148, 150 may be some other suitable portions, such as mating hook and loop type fasteners or fastener portions, buttons, and snap fasteners.

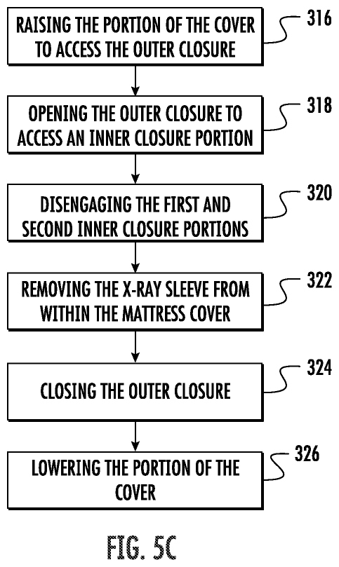

Now with reference to FIG. 5A, there is illustrated a method of using an X-ray cassette within a mattress cover, wherein the method may be comprised of the step of providing a mattress cover having a downwardly extending panel, as indicated in function step 302. At least a portion of the downwardly extending panel is raised to access an inner panel portion of the mattress cover and an outer closure supported by the inner panel portion, as indicated in function step 304, wherein the outer closure may be in the form of a zipper. The outer closure is opened to access an X-ray sleeve within the mattress cover, beneath an upper panel of the mattress cover, as indicated in function step 306. An X-ray cassette is inserted in the X-ray sleeve, as indicated in function step 308. With a patient supported on the mattress cover and properly situated in relation to X-ray equipment, an image of the patient may be produced as desired.

It should be appreciated that, as shown in FIG. 5B, the X-ray sleeve may be releasably attached to the inner panel portion of the top cover by engaging an inner closure portions within an opening in the inner panel portion of the mattress cover, as indicated in function step 310, wherein the inner closure portions may be in the form one or more mating zipper portions that together form one of more zippers. The outer closure may be closed to conceal the inner closure portions, together with the X-ray sleeve, interiorly or inwardly of the inner panel portion and within the mattress cover, as indicated in function step 312. The outer closure may be concealed by lowering the portion of the downwardly extending panel to cover the inner panel portion of the mattress cover and the outer closure supported by the inner panel portion, as indicated in function step 314.

As illustrated in FIG. 5C, the X-ray sleeve is removeable from within the mattress cover by raising the portion of the downwardly extending panel to access the inner panel portion of the mattress cover and the outer closure supported by the inner panel portion, opening the outer closure, disengaging the inner closure portions, removing the X-ray sleeve from within the mattress cover, closing the outer closure, and lowering the portion of the downwardly extending panel to cover the inner panel portion of the mattress cover and the outer closure supported by the inner panel portion, as indicated in function steps 316-326. This permits the X-ray sleeve to be sterilized and sanitized.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

### PARTS LIST

100 mattress cover
102 top cover
104 upper panel
106 laterally extending edges
108 longitudinally extending edges
110 peripheral edge
112 peripheral panel
114 laterally extending panel portions
116 longitudinally extending panel portions
118 inner panel
120 longitudinally extending opening
122 lower peripheral edge
124 longitudinally extending closure
126 upper inner first longitudinally extending closure portion
128 lower inner first longitudinally extending closure portion
130 bottom cover
132 lower panel
134 laterally extending edges
136 longitudinally extending edges
138 peripheral edge
140 peripheral panel
142 laterally extending panel portions
144 longitudinally extending panel portions
146 partial lower enclosure
148 bottom cover closure portion
150 upper peripheral edge
152 top cover closure portion
200 X-ray sleeve
202 upper second longitudinally extending closure portion
204 lower second longitudinally extending closure portion
C X-ray cassette
M mattress

What is claimed is:

1. A mattress cover comprising:
a top cover comprising:
an upper panel defining an upper surface for supporting a patient thereon, the upper panel comprising a peripheral edge,
a peripheral panel depending downwardly in relation to the peripheral edge of the upper panel, the peripheral panel comprising, at least, in part, a longitudinally extending panel portion,
an inner panel depending downwardly in relation to the upper panel inwardly of the peripheral panel, at least a portion of the inner panel defining a longitudinally extending opening supported in relation to at least a portion of the longitudinally extending panel portion,
a longitudinally extending outer closure operable to a closed position to close the opening and an open position to open the opening,
at least a first inner longitudinally extending closure portion within the opening, and
a sleeve extendable laterally beneath the upper panel, the sleeve being dimensioned and configured to receive an X-ray cassette, the sleeve comprising at least a first inner longitudinally extending closure portion that mates with the first inner longitudinally extending closure portion of the top cover to attach the sleeve to the top cover within the opening.

2. The mattress cover of claim 1, wherein the closure is a zipper and the closure portions are zipper portions cooperatively forming a zipper.

3. The mattress cover of claim 1, wherein the top cover further comprises a second inner first longitudinally extending closure portion within the opening and juxtaposed the first inner longitudinally extending closure portion of the top cover, and wherein the sleeve further comprises a second longitudinally extending closure portion that mates with the second inner longitudinally extending closure portion of the top cover to attach the sleeve to the top cover at a second point of contact within the opening.

4. The mattress cover of claim 3, wherein the closure is a zipper and the closure portions are zipper portions.

5. The mattress cover of claim 1, wherein the peripheral panel is pliable so that at least a portion of the peripheral panel is raisable so that at least a portion of the peripheral panel is above the opening to expose the opening, the opening being concealed by the peripheral panel when the peripheral panel extends downwardly from the upper panel.

6. The mattress cover of claim 1, wherein at least one of the top cover and the sleeve is made, at least, in part, of a non-woven breathable, tear-resistant, low-friction, waterproof material.

7. The mattress cover of claim 1, wherein at least one of the top cover and the sleeve is made, at least, in part, of a polyester fabric with a poly-coating.

8. The mattress cover of claim 1, further comprising a mattress enclosable with the mattress cover.

9. The mattress cover of claim 1, further comprising:
a bottom cover comprising:
a lower panel comprising a peripheral edge, and
a peripheral panel extending upwardly in relation to the peripheral edge of the lower panel, the lower panel and the bottom cover peripheral panel cooperatively defining a partial lower enclosure for a mattress,
at least one bottom cover closure portion supported in relation to the bottom cover peripheral panel, and wherein the top cover further comprises at least one top cover closure portion supported in relation to the top cover peripheral panel, the top cover closure portion mating with the bottom cover closure portion to attach the top cover to the bottom cover to cover a mattress therein.

10. The mattress cover of claim 9, wherein the closure is a zipper and each one of the closure portions is a zipper portion.

11. The mattress cover of claim 9, wherein the top cover further comprises a second inner first longitudinally extending closure portion within the opening and juxtaposed the first inner longitudinally extending closure portion of the top cover, and wherein the sleeve further comprises a second longitudinally extending closure portion that mates with the second inner longitudinally extending closure portion of the top cover to attach the sleeve to the top cover at a second point of contact within the opening.

12. The mattress cover of claim 11, wherein the closure is a zipper and the closure portions are zipper portions.

13. The mattress cover of claim 9, wherein the peripheral panel is pliable so that at least a portion of the peripheral panel is raisable so that at least a portion of the peripheral panel is above the opening to expose the opening, the opening being concealed by the peripheral panel when the peripheral panel extends downwardly from the upper panel.

14. The mattress cover of claim 9, wherein at least one of the top cover and the sleeve is made, at least, in part, of a non-woven breathable, tear-resistant, low-friction, waterproof material.

15. The mattress cover of claim 9, wherein at least one of the top cover and the sleeve is made, at least, in part, of a polyester fabric with a poly-coating.

16. The mattress cover of claim 9, further comprising a mattress enclosable with the mattress cover.

17. A mattress cover comprising:
a top cover comprising:
an upper panel defining an upper surface for supporting a patient thereon, the upper panel comprising a peripheral edge,
an upper peripheral panel depending downwardly in relation to the peripheral edge of the upper panel, the upper peripheral panel comprising a lower peripheral edge,
an inner panel depending downwardly in relation to the peripheral edge of the upper panel and inwardly of the upper peripheral panel, the inner panel defining an opening along longitudinally extending portion thereof, the inner panel comprising a lower peripheral edge,
an outer closure operable to a closed position to close the opening and an open position to open the opening,
a bottom cover comprising:
a bottom panel defining a lower surface for supporting a mattress thereon, the bottom panel comprising a peripheral edge, and
a bottom peripheral panel extending upwardly in relation to the peripheral edge of the bottom panel, the bottom panel and the bottom peripheral panel cooperatively defining, at least, in part, a partial lower enclosure for the mattress, the bottom peripheral panel comprising an upper peripheral edge,
at least one peripheral closure for attaching the lower peripheral edge of the inner panel to the upper peripheral edge of the bottom peripheral panel to enclose the mattress within the top and bottom covers, and
a sleeve extendable laterally beneath the upper panel, the sleeve being dimensioned and configured to receive an x-ray cassette, the sleeve being releasably attachable to the inner panel within the opening by at least one matingly engageable closure, where the upper peripheral panel is extendable downwardly to cover the inner panel and the opening defined thereby, as well as the sleeve and the at least one peripheral closure, and the upper peripheral panel being pliable so that at least a portion of the upper peripheral panel is raisable to reveal and provide access to the opening and the sleeve.

18. The mattress cover of claim 17, wherein closures are zippers and the closure portions are zipper portions.

19. The mattress cover of claim 17, wherein at least one of the top cover, the bottom cover and the sleeve is made of a non-woven breathable, tear-resistant, low-friction, waterproof material.

20. The mattress cover of claim 17, further comprising a mattress enclosable with the mattress cover.
